(19) United States
(12) Patent Application Publication
(10) Pub. No.: US 2009/0118237 A1
(43) Pub. Date: May 7, 2009

(54) Carisoprodol, Phenytoin and Fosphenytoin Articles and Methods

(75) Inventors: Richard H. Roberts, Lakewood, NJ (US); Jie Du, Lansdale, PA (US); Matthew W. Davis, Erwinna, PA (US)

Correspondence Address: CANTOR COLBURN, LLP, 20 Church Street, 22nd Floor, Hartford, CT, 06103, US

(73) Assignee: MUTUAL PHARMACEUTICAL COMPANY, INC., Philadelphia, PA (US)

(21) Appl. No.: 11/865,143

(22) Filed: Oct. 1, 2007

Related U.S. Application Data
(60) Provisional application No. 60/827,554, filed on Sep. 29, 2006.

(51) Int. Cl. A61K 31/27; A61K 31/4164; A61K 31/675

(52) U.S. Cl. 514/94; 514/478; 514/385

(57) ABSTRACT

A method of using carisoprodol, phenytoin or fosphenytoin comprises informing a user that co-administration of carisoprodol with phenytoin or fosphenytoin results in an increase in free phenytoin blood levels, a decrease in total phenytoin levels, or both. In another embodiment, a method of using carisoprodol, phenytoin or fosphenytoin comprises informing a user that when co-administering carisoprodol with phenytoin or fosphenytoin, the level of free phenytoin in a patient serum should be monitored, the level of total phenytoin should be monitored, or both. Also included are methods and articles of manufacture.

### CROSS REFERENCE TO RELATED APPLICATION

This application claims priority from U.S. Provisional Application Ser. No. 60/827,554 filed Sep. 29, 2006, which is hereby incorporated by reference in its entirety.

### BACKGROUND

Carisoprodol, chemical name N-isopropyl-2-methyl-2-propyl-1,3-propanediol dicarbamate, is a pharmaceutical active agent whose metabolite is meprobamate. Metabolism of carisoprodol by cytochrome P450 is form CYP2C19 has been reported. Carisoprodol was approved by the U.S. FDA on Apr. 9, 1959. It is marketed in the United States under the brand name Soma®, and in the United Kingdom and other countries under the brand name Carisoma®. Carisoprodol is commonly used as a skeletal muscle relaxant.

Carisoprodol is a colorless, crystalline powder, having a mild, characteristic odor and a bitter taste. It is very sparingly soluble in water and freely soluble in alcohol, chloroform, and acetone.

Phenytoin, 5,5-diphenylhydantoin, is an antiepileptic drug useful in the treatment of epilepsy, which is eliminated via metabolism by cytochrome P450 isoforms, including CYP1A2, CYP2C9, CYP2C19, and CYP3A4. Phenytoin may have beneficial effects in the treatment of trigeminal neuralgia in some patients. Phenytoin has a narrow therapeutic index such that too little can lead to insufficient clinical results and excessive phenytoin can lead to phenytoin toxicity. Physicians typically monitor total phenytoin serum levels. The typical clinically effective serum level of total phenytoin is about 10 to about 20 μg/mL. Phenytoin is highly bound to plasma proteins, yet both the efficacy and toxicity of phenytoin are due to the small fraction of unbound phenytoin. The typical clinically effective serum level of unbound phenytoin is about 1 to about 2 μg/mL. The recommended initial dose is one 100 mg capsule 3 to 4 times per day, with 300 mg/day dose in three divided doses or one single dose per day.

Fosphenytoin, (3-phosphoryloxy-methyl phenytoin disodium), is a prodrug of phenytoin and is completely converted to phenytoin with a half life of about 15 minutes. Fosphenytoin is commercially available as Cerebyx®, a 75 mg/ml solution for parenteral administration. The dose, concentration in dosing solutions, and infusion rate of IV fosphenytoin is expressed as phenytoin sodium equivalents (PE). Fosphenytoin sodium 750 mg is equivalent to 500 mg of phenytoin sodium. Like phenytoin, fosphenytoin is highly bound to serum plasma proteins.

The present invention addresses the need for improved carisoprodol articles and methods of administering carisoprodol.

### SUMMARY

There is an especially important need for improvements in carisoprodol articles and methods because studies of possible negative or competing interactions with narrow therapeutic index drugs have been limited.

In one embodiment, a method of treating a patient in need of a skeletal muscle relaxant comprises administering to a patient in need thereof a composition comprising carisoprodol, and providing to the patient and/or a medical care worker published material providing information that when co-administering carisoprodol with phenytoin or fosphenytoin, the level of free phenytoin in a patient serum sample should be monitored.

A method of preventing overdosing a patient with phenytoin or fosphenytoin comprises co-administering to the patient a dose of carisoprodol and a dose of phenytoin or fosphenytoin, and informing the patient and/or a medical care worker that co-administration of carisoprodol with phenytoin or fosphenytoin can result in an increase in free phenytoin blood levels.

A method of treating a patient in need of treatment for epilepsy comprises providing to the patient in need thereof a dose of phenytoin or fosphenytoin, and a dose of caridsoprodol, and monitoring the free phenytoin concentration in a serum sample from the patient.

In another embodiment, a method of administering carisoprodol comprises co-administering to a patient carisoprodol with phenytoin or fosphenytoin, and monitoring the free phenytoin concentration in a serum sample from the patient.

A method of using carisoprodol, phenytoin or fosphenytoin comprises informing a user that co-administration of carisoprodol with phenytoin or fosphenytoin can result in an increase in free phenytoin blood levels.

These and other embodiments, advantages and features of the present invention become clear when detailed description and examples are provided in subsequent sections.

### DETAILED DESCRIPTION

Carisoprodol is metabolized by cytochrome p450s, including cytochrome p450 isozyme CYP2C19. Other active agents, particularly narrow therapeutic index substances, are also metabolized by cytochrome p450 isozyme CYP2C19. Studies were thus undertaken to determine the effects on plasma concentration, bioavailability, safety, efficacy, or a combination comprising at least one of the foregoing that occur when co-administering carisoprodol and a narrow therapeutic index substance, specifically phenytoin. It was unexpectedly discovered by the inventors herein that the blood levels of free phenytoin were significantly increased when phenytoin was co-administered with carisoprodol. This result is particularly surprising because it has previously been suggested that the effects of phenytoin would be decreased upon co-administration with carisoprodol, not increased as suggested herein. In one embodiment, the increase in free phenytoin occurs whether the phenytoin and carisoprodol are co-administered as a single dose. Such an increase in free phenytoin blood levels can have a significant effect on the safety and/or efficacy of co-administration of carisoprodol with phenytoin or fosphenytoin. It was also unexpectedly discovered by the inventors herein that the blood levels of total phenytoin were significantly decreased when phenytoin was co-administered with carisoprodol in a single-dose study. Such a decrease in total phenytoin blood levels can have a significant effect on the safety and/or efficacy of co-administration of carisoprodol with phenytoin or fosphenytoin.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates), and co-crystals of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts of an active agent) of DRUG or other active agent may be employed either alone or in combination.

“Pharmaceutically acceptable salts” includes derivatives of carisoprodol, wherein the carisoprodol is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the carisoprodol. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

“Efficacy” means the ability of an active agent administered to a patient to produce a therapeutic effect in the patient.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, concomitant medication, or interactions with concomitant medication).

“Enhancing the safety profile” of an active agent means implementing actions or articles designed or intended to help reduce the incidence of adverse events associated with administration of the active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

“Active agent interaction” refers to a change in the metabolism of an active agent in a patient that can occur with co-administration of a second active agent.

Administering an active agent with a substance”, “administering an active agent and a substance”, or “co-administering an active agent and a substance” means the active agent and the substance are administered simultaneously in a single dosage form, administered concomitantly in separate dosage forms, or administered in separate dosage forms separated by some amount of time that is within the time in which both the active agent and the substance are within the blood stream of a patient. The active agent and the substance need not be prescribed for a patient by the same medical care worker. Administration of the active agent or the substance can occur via any appropriate route, for example, oral tablets, oral capsules, oral liquids, inhalation, injection, suppositories or topical contact.

“Adverse event” means any untoward medical occurrence in a patient administered an active agent and which does not necessarily have to have a causal relationship with this treatment. An adverse event (AE) can therefore be any unfavorable and unintended sign (including an abnormal laboratory finding, for example), symptom, or disease temporally associated with the use of the active agent, whether or not considered related to the active agent.

“Adverse reaction” means a response to an active agent which is noxious and unintended and which occurs at doses normally used in humans for prophylaxis, diagnosis, or therapy of disease or for modification of physiological function. The unintended response can be an unexpected diminished or enhanced pharmacologic activity or toxicity of the active agent. An adverse reaction also includes any undesirable or unexpected event requiring discontinuation of the active agent, modification of the dose, prolonged hospitalization, or the administration of supportive treatment.

“Affects” include an increase or decrease in degree, level, or intensity; a change in time of onset or duration; a change in type, kind, or effect, or a combination comprising at least one of the foregoing.

“Dosing regimen” means the dose of an active agent taken at a first time by a patient and the interval (time or symptomatic) at which any subsequent doses of the active agent are taken by the patient. The additional doses of the active agent can be different from the dose taken at the first time.

A “dose” means the measured quantity of an active agent to be taken at one time by a patient.

Carisoprodol is a “very sparingly soluble” compound, having a solubility in water of 0.3 to 1.4 mg/mL between 25 to 50° C.

A substance having a “narrow therapeutic index” (NTI) means a substance falling within any definition of narrow therapeutic index as promulgated by the U.S. Food and Drug Administration or any successor agency thereof, for example, a substance having a less than 2-fold difference in median lethal dose (LD50) and median effective dose (ED50) values for the substance, or having a less than 2-fold difference in the minimum toxic concentration and minimum effective concentration in the blood of the substance.

A “dosage form” means a unit of administration of an active agent. Examples of dosage forms include tablets, capsules, injections, suspensions, liquids, emulsions, creams, ointments, suppositories, inhalable forms, transdermal forms, and the like.

The term “effective amount” or “therapeutically effective amount” means an amount effective, when administered to a patient, to provide any therapeutic benefit. A therapeutic benefit may be an amelioration of symptoms, e.g., an amount effective to decrease the symptoms of epilepsy for phenytoin and fosphenytoin. A therapeutic benefit may be an amount suitable to acts as a skeletal muscle relaxant for carisoprodol. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation. In certain circumstances a patient may not present symptoms of a condition for which the patient is being treated. A therapeutically effective amount of an active agent may also be an amount sufficient to provide a significant positive effect on any indicium of a disease, disorder, or condition. A significant effect on an indicium of a disease, disorder, or condition is statistically significant in a standard parametric test of statistical significance, for example Student's T-test, where p≦0.05.

By “oral dosage form” is meant to include a unit dosage form for oral administration. An oral dosage form may optionally comprise a plurality of subunits such as, for example, microcapsules or microtablets. Multiple subunits may be packaged for administration in a single dose. By “subunit” is meant to include a composition, mixture, particle, pellet, etc., that can provide an oral dosage form alone or when combined with other subunits.

A dissolution profile is a plot of the cumulative amount of active agent released as a function of time. A dissolution profile can be measured utilizing the Drug Release Test <724>, which incorporates standard test USP 26 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate-release and controlled-release characteristics, for example, a combination of immediate-release pellets and controlled-release pellets. The immediate-release portion of a combination dosage form may be referred to as a loading dose.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

Under U.S. FDA guidelines, two products or methods (e.g., dosing under non-fasted versus fasted conditions) are bioequivalent if the 90% Confidence Intervals (CI) for a log transformed geometric mean of AUC_{0-INF}, and Cₘₐₓ are 0.80 to 1.25 (Tₘₐₓ measurements are not relevant to bioequivalence for regulatory purposes). To show bioequivalency between two compounds or administration conditions pursuant to Europe's EMEA guidelines, the 90% CI for a log transformed geometric mean of AUC_{0-INF}, must be 0.80 to 1.25 and the 90% CI for a log transformed geometric mean of Cₘₐₓ must be 0.70 to 1.43.

Information as disclosed herein may include information that two administration methods are bioequivalent under FDA guidelines, are substantially bioequivalent, or have insignificant differences in their pharmacokinetic parameters.

Certain formulations described herein may be “coated”. The coating may be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

“Informing” means referring to or providing, published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

“Labeling” means all labels or other means of written, printed, graphic, electronic, verbal, or demonstrative communication that is upon a pharmaceutical product or a dosage form or accompanying such pharmaceutical product or dosage form.

A “medical care worker” means a worker in the health care field who may need or utilize information regarding an active agent including a dosage form thereof, including information on safety, efficacy, dosing, administration, or pharmacokinetics. Examples of medical workers include physicians, pharmacists, physician's assistants, nurses, aides, caretakers (which can include family members or guardians), emergency medical workers, and veterinarians.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

A “pharmaceutical supplier” means a person (other than a medical care worker), business, charitable organization, governmental organization, or other entity involved in the transfer of active agent, including a dosage form thereof, between entities, for profit or not. Examples of pharmaceutical suppliers include pharmaceutical distributors, pharmacy chains, pharmacies (online or physical), hospitals, HMOs, supermarkets, the Veterans Administration, or foreign businesses or individuals importing active agent into the United States.

A “user” means a patient, a medical care worker, or a pharmaceutical supplier.

A “product” or “pharmaceutical product” means a dosage form of an active agent plus published material and optionally packaging.

“Providing” means giving, administering, selling, distributing, transferring (for profit or not), manufacturing, compounding, or dispensing.

“Published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

“Product insert” means the professional labeling (prescribing information) for a pharmaceutical product, a patient package insert for the pharmaceutical product, or a medication guide for the pharmaceutical product.

“Professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

“Patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

“Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

Food typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria. In one embodiment, with food means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

Carisoprodol is metabolized by cytochrome p450s, including cytochrome p450 isozyme CYP2C19. Other active agents, particularly narrow therapeutic index substances, are also metabolized by cytochrome p450 isozyme CYP2C 19. Studies were thus undertaken to determine the effects on plasma concentration, bioavailability, safety, efficacy, or a combination comprising at least one of the foregoing that occur when co-administering carisoprodol and a narrow therapeutic index substance, specifically phenytoin. Because fosphenytoin is a prodrug of phenytoin, similar results are expected for fosphenytoin

Phenytoin is eliminated via metabolism by cytochrome P450 isoforms, including CYP1A2, CYP2C9, CYP2C19, and CYP3A4. Phenytoin is highly bound (e.g., about 90% bound) to plasma proteins in the bloodstream such as albumin. For phenytoin, drug response is dependent on free drug concentration, that is, the about 10% of the drug that is not bound to plasma proteins. Since it is the free phenytoin in plasma which equilibrates with the site of pharmacological or toxic response, a slight change in the extent of binding leads to a substantial percentage change in free phenytoin concentration, which in turn causes a very significant alteration in response. Slight changes in the binding of highly bound drugs to plasma proteins leads to significant changes in the clinical response, and in some cases causes a toxic response.

Similarly, fosphenytoin, a prodrug of phenytoin, is highly bound to plasma proteins. Also, because fosphenytoin is metabolized to phenytoin, the clinical results for phenytoin are extended to administration of fosphenytoin.

Due to the narrow therapeutic index of phenytoin and fosphenytoin, plus wide individual variability in the rate of phenytoin metabolism and clearance, therapeutic drug monitoring during treatment with phenytoin or fosphenytoin is typically recommended. Insufficient blood levels may lead to inadequate symptom management, while excess blood levels can lead to dangerous toxicity. However, because the measurement of total phenytoin blood levels is easier than the measurement of free phenytoin blood levels, physicians typically monitor total phenytoin blood levels.

In one embodiment, monitoring the patient comprises monitoring the patient's plasma concentration of phenytoin, specifically free phenytoin; monitoring the patient for symptoms of an active agent interaction between carisoprodol and phenytoin; monitoring the patient for an adverse reaction (e.g., toxicity) resulting from administration of carisoprodol and phenytoin; monitoring the patient for an adverse reaction (e.g., toxicity) resulting from administration of phenytoin; or monitoring the patient for decreased efficacy of phenytoin. Monitoring is performed, for example, daily in the early stages of treatment, to once yearly once a treatment protocol is established. In one embodiment, monitoring is performed once every 7 to 14 days.

Total phenytoin in a serum sample can be measured, for example, in a turbidimetry assay. Turbidimetry measures the diminution in power of a collimated light beam as a result of scattering particles in solution, measured for example in a spectrophotometer. The principle of the method is the competition of phenytoin in a serum sample with phenytoin labeled latex particles for anti-phenytoin antibodies. Phenytoin bound to latex reacts with anti-phenytoin antibodies to form immune complexes in an appropriate buffer. Phenytoin in a serum sample competes with phenytoin-latex and this competitive reaction can be measured by turbidimetry. The absorbance is inversely related to the phenytoin concentration in the serum sample. The actual concentration is determined from a calibration curve prepared with standards of known concentration.

Free phenytoin in a serum sample can be measured, for example, in a fluorescence polarization immunoassay (FPIA). First, protein-bound phenytoin is separated from free phenytoin using, for example, ultrafiltration. Then the isolated free phenytoin is measured by FPIA. FPIA takes advantage of the increased polarization non-random propagation of emission of fluorescent light emissions when a fluorescently labeled antigen is bound by reagent antibody. The higher the concentration of unlabeled patient phenytoin in the test mixture, the less bound fluorescent phenytoin is present and, consequently, the lower the polarization of the fluorescent light emission. Standard calibration yields quantitative results.

It has been unexpectedly discovered that co-administration of carisoprodol with phenytoin in a single-dose study leads to a statistically significant increase in free phenytoin blood levels. Concurrently, there is a decrease in total phenytoin blood levels in a single-dose co-administration study. Such a change is clinically significant as small changes in free phenytoin blood levels can lead to significant changes in clinical response and in some cases causes a toxic response. Also, the lower level of total phenytoin may cause a physician to erroneously infer that free phenytoin has also decreased.

In one embodiment, a method of using carisoprodol comprises informing a user that co-administration of carisoprodol with phenytoin or fosphenytoin results in an increase in free phenytoin blood levels. In another embodiment, co-administration of carisoprodol with phenytoin or fosphenytoin results in a decrease in total phenytoin blood levels. The co-administration can lead to changes in the safety and/or efficacy of the carisoprodol, phenytoin, fosphenytoin, or all three. Changes in safety and/or efficacy include changes in clinical response and/or a toxic response. When co-administering carisoprodol with phenytoin or fosphenytoin, the level of free phenytoin in a patient serum should be monitored, the level of total phenytoin in a patient serum should be monitored, or both. In one embodiment, the level of free phenytoin is measured. In another embodiment, a method of using carisoprodol comprises informing a user that when co-administering carisoprodol with phenytoin or fosphenytoin, the level of free phenytoin in a patient serum should be monitored, the level of total phenytoin in a patient serum should be monitored, or both.

In one embodiment, a method of using carisoprodol, comprises obtaining carisoprodol from a container providing information that when co-administering carisoprodol with phenytoin or fosphenytoin, the level of free phenytoin in a patient serum should be monitored, the level of total phenytoin in a patient serum should be monitored, or both. Specifically, the level of free phenytoin in a patient serum should be monitored. In another embodiment, a method of manufacturing a carisoprodol pharmaceutical composition comprises packaging a carisoprodol pharmaceutical formulation along with information that when co-administering carisoprodol with phenytoin or fosphenytoin, the level of free phenytoin in a patient serum should be monitored, the level of total phenytoin in a patient serum should be monitored, or both. In yet another embodiment, an article of manufacture comprises a container holding a dosage form of carisoprodol, wherein the container is associated with printed labeling instructions advising that when co-administering carisoprodol with phenytoin or fosphenytoin, the level of free phenytoin in a patient serum should be monitored, the level of total phenytoin in a patient serum should be monitored, or both.

In one embodiment, a method of using phenytoin comprises informing a user that co-administration of carisoprodol with phenytoin can change the safety and/or efficacy of phenytoin. Changes in safety and/or efficacy include changes in clinical response and/or a toxic response. In another embodiment, a method of using phenytoin comprises informing a user that co-administration of carisoprodol with phenytoin results in an increase in free phenytoin blood levels, a decrease in total phenytoin blood levels, or both, which can change the safety and/or efficacy of phenytoin. Because it is the free phenytoin that causes both the therapeutic and toxic effects of phenytoin, when co-administering phenytoin with carisoprodol, the level of free phenytoin in a patient serum should be monitored, the level of total phenytoin in a patient serum should be monitored, or both. Thus, in another embodiment, a method of using phenytoin comprises informing a user that when co-administering carisoprodol with phenytoin, the level of free phenytoin in a patient serum should be monitored the level of total phenytoin in a patient serum should be monitored, or both.

In one embodiment, a method of using phenytoin comprises obtaining phenytoin from a container providing information that when co-administering carisoprodol with phenytoin, the level of free phenytoin in a patient serum should be monitored, the level of total phenytoin in a patient serum should be monitored, or both. In another embodiment, a method of manufacturing a phenytoin pharmaceutical composition comprises packaging a phenytoin pharmaceutical formulation along with information that when co-administering carisoprodol with phenytoin, the level of free phenytoin in a patient serum should be monitored, the level of total phenytoin in a patient serum should be monitored, or both. In yet another embodiment, an article of manufacture comprises a container holding a dosage form of phenytoin, wherein the container is associated with printed labeling instructions advising that when co-administering carisoprodol with phenytoin, the level of free phenytoin in a patient serum should be monitored the level of total phenytoin in a patient serum should be monitored, or both.

In one embodiment, a method of using fosphenytoin comprises informing a user that co-administration of carisoprodol with fosphenytoin can change the safety and/or efficacy of fosphenytoin. Changes in safety and/or efficacy include changes in clinical response and/or a toxic response. In another embodiment, a method of using fosphenytoin comprises informing a user that co-administration of carisoprodol with fosphenytoin results in an increase in free phenytoin blood levels, a decrease in total phenytoin blood levels, or both, which can change the safety and/or efficacy of fosphenytoin. Because it is the free phenytoin that causes both the therapeutic and toxic effects of phenytoin, when co-administering fosphenytoin with carisoprodol, the level of free phenytoin in a patient serum should be monitored, the level of total phenytoin in a patient serum should be monitored, or both. Thus, in another embodiment, a method of using fosphenytoin comprises informing a user that when co-administering carisoprodol with fosphenytoin, the level of free phenytoin in a patient serum should be monitored the level of total phenytoin in a patient serum should be monitored, or both.

In one embodiment, a method of using fosphenytoin comprises obtaining fosphenytoin from a container providing information that when co-administering carisoprodol with fosphenytoin, the level of free phenytoin in a patient serum should be monitored, the level of total phenytoin in a patient serum should be monitored, or both. In another embodiment, a method of manufacturing a fosphenytoin pharmaceutical composition comprises packaging a fosphenytoin pharmaceutical formulation along with information that when co-administering carisoprodol with fosphenytoin, the level of free phenytoin in a patient serum should be monitored, the level of total phenytoin in a patient serum should be monitored, or both. In yet another embodiment, an article of manufacture comprises a container holding a dosage form of fosphenytoin, wherein the container is associated with printed labeling instructions advising that when co-administering carisoprodol with fosphenytoin, the level of free phenytoin in a patient serum should be monitored the level of total phenytoin in a patient serum should be monitored, or both.

In one embodiment, informing comprises providing information that single-dose co-administration of phenytoin and carisoprodol resulted in a 14% increase in the Cₘₐₓ for free phenytoin and an 8% increase in the AUC₀₋ₜ for free phenytoin.

In one embodiment, the published material informs that, in a single-dose co-administration study, the geometric mean of Cₘₐₓ for free phenytoin when phenytoin is administered alone is not bioequivalent to that when phenytoin is co-administered with carisoprodol. The published material optionally further informs that the geometric mean of Cₘₐₓ for total phenytoin when phenytoin is administered alone is bioequivalent to that when phenytoin is co-administered with carisoprodol.

In another embodiment, the published material informs that in a single-dose co-administration study, co-administration of phenytoin with carisoprodol results in an increase in Cₘₐₓ for free phenytoin. The published material optionally further informs that co-administration of phenytoin with carisoprodol results in a decrease in Cₘₐₓ for total phenytoin.

In some embodiments, the published material informs that that in a in a single-dose co-administration study of 12 patients, the geometric mean of Cₘₐₓ for total phenytoin when phenytoin was administered alone was 3222.2 ng/ml, and the geometric mean of Cₘₐₓ for total phenytoin when phenytoin was co-administered with carisoprodol was 2801.38 ng/ml. In other embodiments, the published material informs that that in a in a single-dose co-administration study of 12 patients, the geometric mean of Cₘₐₓ for free phenytoin when phenytoin was administered alone was 282.76 ng/ml, and the geometric mean of Cₘₐₓ for free phenytoin when phenytoin was co-administered with carisoprodol was 322.25 ng/ml. In one embodiment, the printed material informs that in a single-dose co-administration study, the lower limit of the 90% confidence interval of the ratio of Cₘₐₓ for free phenytoin when phenytoin was administered alone to free phenytoin when phenytoin was co-administered with carisoprodol was 69.5%.

In one embodiment, the published material informs that in a single-dose co-administration study, the geometric mean of AUC₀₋ₜ for free phenytoin when phenytoin is administered alone is not bioequivalent to that when phenytoin is co-administered with carisoprodol. The published material optionally further informs that in a single-dose co-administration study, the geometric mean of AUC₀₋ₜ for total phenytoin when phenytoin is administered alone is bioequivalent to that when phenytoin is co-administered with carisoprodol.

In another embodiment, the published material informs that co-administration of phenytoin with carisoprodol results in an increase in AUC₀₋ₜ for free phenytoin. The published material optionally further informs that co-administration of phenytoin with carisoprodol in a single-dose co-administration study results in a decrease in AUC₀₋ₜ for total phenytoin.

In one embodiment, the published material informs that the geometric mean of AUC₀₋ₜ for total and/or free phenytoin when phenytoin is administered alone is bioequivalent to that when phenytoin is co-administered with carisoprodol. In some embodiments, the published material informs that that in a study of 12 patients, the geometric mean of AUC₀₋ₜ for total phenytoin when phenytoin was administered alone was 97342.7 hr*ng/ml, and the geometric mean of AUC₀₋ₜ for total phenytoin when phenytoin was co-administered with carisoprodol was 91468.8 hr*ng/ml. In some embodiments, the published material informs that that in a study of 12 patients, the geometric mean of AUC₀₋ₜ for free phenytoin when phenytoin was administered alone was 5150.25 hr*ng/ml, and the geometric mean of AUC₀₋ₜ for free phenytoin when phenytoin was co-administered with carisoprodol was 5559.62 hr*ng/ml. In one embodiment, the printed material informs that the lower limit of the 90% confidence interval of the ratio of AUC₀₋ₜ for free phenytoin when phenytoin is administered alone to free phenytoin when phenytoin is co-administered with carisoprodol is 75.34%.

In one embodiment, the published material informs that the geometric mean of AUC_{0-INF} for total and/or free phenytoin when phenytoin was administered alone is bioequivalent to that when phenytoin was co-administered with carisoprodol. In some embodiments, the published material informs that that in a study of 12 patients, the geometric mean of AUC_{0-INF} for total phenytoin when phenytoin was administered alone was 133987.2 hr*ng/ml, and the geometric mean of AUC_{0-INF} for total phenytoin when phenytoin was co-administered with carisoprodol was 132006.5 hr*ng/ml. In some embodiments, the published material informs that that in a study of 12 patients, the geometric mean of AUC_{0-INF} for free phenytoin when phenytoin was administered alone was 12927.88 hr*ng/ml, and the geometric mean of AUC_{0-INF} for free phenytoin when phenytoin was co-administered with carisoprodol was 100673.26 hr*ng/ml.

In the foregoing embodiments, the methods optionally further comprise administering providing the user carisoprodol, phenytoin and/or fosphenytoin.

The informing is, for example, by reference to published material; by reference to a package active agent insert, a flyer or an advertisement; by presentation of information at a seminar, conference, or other educational presentation; or by a conversation between a pharmaceutical sales representative and the medical care worker. Informing comprises, for example, providing published material comprising a discussion that when co-administering carisoprodol and phenytoin, the level of free phenytoin in a patient serum should be monitored.

Solid dosage forms for oral administration include, but are not limited to, capsules, tablets, pills, powders, and granules. In such solid dosage forms, the active agent may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules, tablets, and pills, the dosage forms may also comprise buffering agents.

The dosage forms described herein may be coated with a functional or non-functional coating. The coating may comprise about 0 wt % to about 40 wt % of the composition. The coating material may include a polymer, such as a film-forming polymer including, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly(ethyl methacrylate), poly(butyl methacrylate), poly(isobutyl methacrylate), poly(hexyl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, (poly propylene), poly(ethylene glycol poly (ethylene oxide), poly(ethylene terephthalate), poly(vinyl alcohol), poly(vinyl isobutyl ether), poly(vinyl acetate), poly(vinyl chloride), polyvinyl pyrrolidone, and combinations comprising one or more of the foregoing polymers.

In some applications, the polymer can be a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having, for example, a low quaternary ammonium content, and the like, and combinations comprising one or more of the foregoing polymers.

In controlled-release applications, for example, the coating can be a hydrophobic polymer that modifies the release properties of the API from the formulation. Suitable hydrophobic or water insoluble polymers for controlled-release include, for example, methacrylic acid esters, ethyl cellulose, cellulose acetate, polyvinyl alcohol-maleic anhydride copolymers, β-pinene polymers, glyceryl esters of wood resins, and combinations comprising one or more of the foregoing polymers.

The inclusion of an effective amount of a plasticizer in the coating composition may improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 wt % to about 50 wt % of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and combinations comprising one or more of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and combinations comprising one or more of the foregoing plasticizers, although it is possible that other plasticizers (such as acetylated monoglycerides, phthalate esters, castor oil, etc.) can be used.

In certain embodiments, it is preferred that the coating is a substantially continuous coat and substantially hole-free. By substantially continuous coating is meant a coating which retains a smooth and continuous appearance when magnified 1000 times under a scanning electron microscope and wherein no holes or breakage of the coating are evident.

Suitable methods can be used to apply the coating to the dosage form. Processes such as simple or complex coacervation, interfacial polymerization, liquid drying, thermal and ionic gelation, spray drying, spray chilling, fluidized bed coating, pan coating, or electrostatic deposition, may be used. A substantially continuous nature of the coating may be achieved, for example, by spray drying from a suspension or dispersion of the coating composition.

The coatings may be about 0.005 micrometers to about 25 micrometers thick, preferably about 0.05 micrometers to about 5 micrometers.

In one embodiment, a carisoprodol dosage form is an oral dosage form such as, for example, a tablet. Oral dosage forms comprise about 100 mg to about 1000 mg of carisoprodol, specifically about 200 mg to about 400 mg of carisoprodol, and more specifically about 350 mg of carisoprodol. In one embodiment, the oral dosage form is an immediate-release oral dosage form.

In one embodiment, a phenytoin dosage form is an oral dosage form such as, for example, a tablet. Oral dosage forms comprise about 20 mg to about 500 mg of phenytoin, specifically about 50 mg to about 200 mg of phenytoin, and more specifically about 100 mg of phenytoin. In one embodiment, the oral dosage form is an immediate-release oral dosage form.

### EXAMPLES

### Example 1

### Study of Phenytoin Pharmacokinetics when Phenytoin is Administered Alone or is Co-Administered with Carisoprodol as a Single-Dose

The study was designed as a randomized, single-dose two-way crossover to compare the pharmacokinetic parameters of carisoprodol (350 mg SOMA® (carisoprodol) Tablets by MedPointe Pharmaceuticals, MedPointe Healthcare Inc.) and phenytoin (PROMPT® Phenytoin Sodium 100 mg Capsules by IVAX Pharmaceuticals, Inc.). Twelve healthy adults participated in this comparison study and all of the subjects completed the study.

Subjects received two separate drug administration treatments in assigned periods, one treatment per period, according to the randomization schedule. Treatment (A) was 3 PROMPT® Phenytoin Sodium 100 mg capsules with 240 mL of room temperature water after an overnight fast. Treatment (B) was 3 PROMPT® Phenytoin Sodium 100 mg capsules and 2 tablets of SOMA® (carisoprodol 350 mg tablets) after an overnight fast.

Dosing days were separated by a washout period of at least fourteen days. Blood samples were drawn prior to dosing (pre-dose) and at 0.5, 1, 1.5, 2, 2.5, 3, 3.5, 4, 4.5, 5, 6, 7, 8, 10, 12, 18, 24, and 48 hours post-dose. The samples were then analyzed for total phenytoin. At study hours 2, 4, 6, 12 and 24 an additional blood sample was collected for the determination of “free” phenytoin.

The following pharmacokinetic parameters may be determined from the plasma concentration data:

The area under the plasma concentration versus time curve [AUCₜ] may be calculated using the linear trapezoidal rule from the zero time point to the last measured concentration.

The area under the plasma concentration versus time curve from zero to infinity [AUC_{0-INF}] may be calculated by adding Cₜ/Kₑₗₘ to AUC where Cₜ is the last measured concentration and Kₑₗₘ is the elimination rate constant.

The maximum observed plasma concentration [Cₘₐₓ] may be obtained by inspection.

The time to maximum plasma concentration [Tₘₐₓ] may be obtained by inspection. If the same maximum plasma concentration occurs at more than one time point, the first may be chosen as Tₘₐₓ.

The terminal elimination rate constant [Kₑₗₘ] may be obtained from the slope of the line, fitted by linear least squares regression, through the terminal points of the ln(base e) of the concentration versus time plot for these points.

The half-life [T_{1/2}] may be calculated by the equation T_{1/2}=0.693/Kₑₗₘ.

The data for free phenytoin when co-administered with carisoprodol is shown in Tables 1 and 2:

TABLE 1
Ln-transformed pharmacokinetic parameters for free
phenytoin when co-administered with carisoprodol

| | | | | 90% |
|---|---|---|---|---|
| | Phenytoin | | | Confidence |
| | alone, | Phenytoin + | | Interval |
| | Geometric | Carisoprodol, | | (Lower limit, |
| | Mean | Geometric Mean | % Ratio | upper limit) |
| Cₘₐₓ | 282.76 | 322.25 | 87.75 | (69.5, 110.79) |
| (ng/ml) | | | | |
| AUC₀₋ₜ | 5150.25 | 5559.62 | 92.64 | (75.34, 113.9) |
| (hr*ng/ml) | | | | |
| AUC_{0-INF} | 12927.88 | 100673.26 | 121.12 | (0, 0) |
| (hr*ng/ml) | | | | |

TABLE 2
Non-transformed pharmacokinetic parameters for free
phenytoin when co-administered with carisoprodol

| | Phenytoin | | |
|---|---|---|---|
| | alone, | Phenytoin + | |
| | Least Sq. | Carisoprodol, | |
| | Mean | Least Sq. Mean | % Ratio |
| Tₘₐₓ (hr) | 9.17 | 8.68 | 105.6 |
| kₑₗₘ | 0.0279 | 0.0361 | 77.18 |
| T_{1/2} (hr) | 35.82 | 22.94 | 156.12 |

Single-dose co-administration of phenytoin and carisoprodol resulted in a 14% increase in the Cₘₐₓ for free phenytoin and an 8% increase in the AUC₀₋ₜ for free phenytoin.

The data for total phenytoin when co-administered with carisoprodol is shown in Tables 3 and 4:

TABLE 3
Ln-transformed pharmacokinetic parameters for total
phenytoin when co-administered with carisoprodol

| | | | | 90% |
|---|---|---|---|---|
| | Phenytoin | | | Confidence |
| | alone, | Phenytoin + | | Interval |
| | Geometric | Carisoprodol, | | (Lower limit, |
| | Mean | Geometric Mean | % Ratio | upper limit) |
| Cₘₐₓ | 3222.2 | 2801.38 | 115.0 | (106.1, 124.7) |
| (ng/ml) | | | | |
| AUC₀₋ₜ | 97342.7 | 91468.8 | 106.4 | (96.35, 117.55) |
| (hr* ng/ml) | | | | |
| AUC_{0-INF} | 133987.2 | 132006.5 | 101.5 | (89.71, 114.84) |
| (hr*ng/ml) | | | | |

TABLE 4
Non-transformed pharmacokinetic parameters for total
phenytoin when co-administered with carisoprodol

| | Phenytoin | | |
|---|---|---|---|
| | alone, | Phenytoin + | |
| | Least Sq. | Carisoprodol, | |
| | Mean | Least Sq. Mean | % Ratio |
| Tₘₐₓ (hr) | 7.437 | 7.429 | 100.11 |
| kₑₗₘ | 0.03395 | 0.033833 | 100.35 |
| T_{1/2} (hr) | 23.91 | 27.08 | 88.29 |

Single-dose co-administration of phenytoin and carisoprodol resulted in a 13% increase in the Cₘₐₓ for total phenytoin, a 6% increase in the AUC₀₋ₜ for total phenytoin, and a 1.5% increase in the AUC_{0-INF} for total phenytoin.

The conclusion from these data is that when phenytoin and carisoprodol are co-administered, the Cₘₐₓ and AUC₀₋ₜ for free phenytoin increases when compared to administration of phenytoin alone. The Cₘₐₓ and AUC₀₋ₜ for free phenytoin co-administered with carisoprodol is not bioequivalent to phenytoin administered alone. In addition, the Cₘₐₓ and AUC₀₋ₜ for total phenytoin decrease somewhat when phenytoin is co-administered with carisoprodol. Thus, when co-administering carisoprodol and phenytoin, the blood level of free phenytoin should be measured.

The terms “a” and “an” do not denote a limitation of quantity, but rather denote the presence of at least one of the referenced item.

The term “or” means “and/or”.

The terms “comprising”, “having”, “including”, and “containing” are to be construed as open-ended terms (i.e., meaning “including, but not limited to”).

The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating a patient in need of a skeletal muscle relaxant, comprising:
administering to a patient in need thereof a composition comprising carisoprodol, and
providing to the patient and/or a medical care worker published material providing information that when co-administering carisoprodol with phenytoin or fosphenytoin, the level of free phenytoin in a patient serum sample should be monitored.

2. The method of claim 1, wherein the information provides that the level of free phenytoin in the patient serum is measured once every 7 to 14 days.

3. The method of claim 1, further comprising determining if the patient is also co-administered phenytoin or fosphenytoin with the carisoprodol.

4. The method of claim 2, further comprising monitoring the free phenytoin concentration in a serum sample from the patient when the patient is co-administered phenytoin or fosphenytoin with the carisoprodol.

5. The method of claim 1, wherein the published material includes information that single-dose, co-administration of phenytoin and carisoprodol resulted in a 14% increase in the Cₘₐₓ for free phenytoin and an 8% increase in the AUC₀₋ₜ for free phenytoin.

6. The method of claim 1, wherein the published material includes information that in a single-dose co-administration study of 12 patients,
the geometric mean of Cₘₐₓ for free phenytoin when phenytoin was administered alone was 282.76 ng/ml, and the geometric mean of Cₘₐₓ for free phenytoin when phenytoin was co-administered with carisoprodol was 322.25 ng/ml; and
the geometric mean of AUC₀₋ₜ for free phenytoin when phenytoin was administered alone was 5150.25 hr*ng/ml, and the geometric mean of AUC₀₋ₜ for free phenytoin when phenytoin was co-administered with carisoprodol was 5559.62 hr*ng/ml.

7. The method of claim 5, further comprising providing information that single-dose, co-administration of phenytoin and carisoprodol resulted in a 13% increase in the Cₘₐₓ for total phenytoin and a 6% increase in the AUC₀₋ₜ for total phenytoin.

8-9. (canceled)

10. The method of claim 16, wherein the free phenytoin in the serum sample from the patient is measured once every 7 to 14 days.

11-13. (canceled)

14. A method of treating a patient in need of treatment for epilepsy, comprising:
providing to a patient in need thereof a dose of phenytoin or fosphenytoin, and a dose of caridsoprodol, and
monitoring the free phenytoin concentration in a serum sample from the patient.

15. The method of claim 14, wherein the free phenytoin in the serum sample from the patient is measured is measured once every 7 to 14 days.

16. A method of administering carisoprodol, comprising
co-administering to a patient carisoprodol with phenytoin or fosphenytoin, and
monitoring the free phenytoin concentration in a serum sample from the patient.

17. The method of claim 16, further comprising
monitoring the total phenytoin level in the serum sample from the patient.

18. The method of claim 16, further comprising adjusting the dosage of phenytoin or fosphenytoin in response to the monitoring.

19-20. (canceled)

21. The method of claim 14, further comprising
monitoring the total phenytoin level in the serum sample from the patient.
